(19) United States
(12) Patent Application Publication
(10) Pub. No.: US 2005/0222654 A1
(43) Pub. Date: Oct. 6, 2005

(54) Facial harness for supporting a hot-cold pack

(76) Inventor: Michael D. Brown, Lakewood, CA (US)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/817,101

(22) Filed: Apr. 1, 2004

(51) Int. Cl. A61F 7/00

(52) U.S. Cl. 607/109; 607/114

(57) ABSTRACT

A facial harness extends in contact with a portion of the human face and neck. An approximately rectangular fabric pocket extends between an upper strap, a lower strap and left and right opposing side straps. The pockets are in contact with the lower sides of the face and receive hot-cold packs for therapeutic contact with the face. A pair of straps hold the pockets in place on the face and neck. Horizontal and vertical oriented pockets are appropriately placed for thermal contact with certain muscles in the face.

### INCORPORATION BY REFERENCE

Applicant(s) hereby incorporate herein by reference, any and all U.S. patents and U.S. patent applications cited or referred to in this application.

### BACKGROUND OF THE INVENTION

1. Field of the Invention

This invention relates generally to head mounted supports and more particularly to such a support having advantages for holding an ice pack along the exterior jaw adjacent to the teeth and jaw bone.

2. Description of Related Art

The following art defines the present state of this field:

Bagg et al., U.S. Des. 290,512 describes an ice bag holder design.

Baker, U.S. Pat. No. 3,491,761 describes a thermal applicator body harness for versatile application of thermal packs to an area of treatment on the human body comprising a thermal pack having a filling throat with an annualar neck on said thermal pack, a removable closure to seal said throat, a harness band of pliable gauze type material having a series of annular openings to slightly stretch to selectively receive said annular neck in snug engagement therewith and clamped to said harness band by said closure, and a pair of strap means secured to said harness band in spaced relation to each other to stabilize and retain said thermal pack on a selected position on the person of the wearer.

Brennan, U.S. Pat. No. 4,190,054 describes a removable bandage attachable to a body part for entirely covering same, the bandage being made from an elastic material so as to hold the flesh of the body part firmly in place following surgery, a plurality of Velcro strips positioned on the outside surface of the bandage, a flexible fluid retaining envelope having a fluid therein which is heatable or coolable so that the envelope functions as a hot or cold pack, complementary Velcro strips on one side of the envelope to permit attachment of the envelope to the bandage without moving the bandage out of contact with the body part, and a layer of insulating material covering the outside of the envelope for reducing heat loss therefrom.

Keen, U.S. Pat. No. 5,020,536 describes a postoperative cryokinetic therapy apparatus for selectively positioning a cold pack such as ice on the face of a patient following oral and maxillofacial surgery and for retaining the cold pack in the selected position during the patient's exercise of the jaw muscles. The apparatus comprises two vertical and one horizontal strap selectively adjustable and secured by velcro to facilitate the positioning of the cold pack. The vertical strap contains pouches on the patients facing side thereof for removably receiving sealable containers for ice or the like.

Hubbard et al., U.S. Pat. No. 5,109,841 describes an ice pack which comprises a waterproof envelope having a first and second side, and further having a sealable open end and a closed end. A strap is attached at one end to the waterproof envelope and has a centrally disposed longitudinal slit. A fastener is in turn attached to the free end of the strap, and is fastenable to an outer surface of the waterproof envelope.

Leo et al., U.S. Pat. No. 6,451,046 describes a device for holding an icepack or other heat transfer means on various parts of the face of a user including an elastic headband strap adjustably secured by hook and pile interconnection, and a second strap which interconnects with the headband at both ends with hook and pile interconnection, which supports the icepack.

Momtaheni, U.S. Pat. No. 6,562,060 describes a device to treat the temporomandibular and maxillomandibular region of patient with cryo, thermal and/or compression therapy. The device comprises a fabric layer anatomically designed to contact the temporomandibular joint and the maxillomandibular region, an inflatable layer, and a heater for heating the masticatory muscles, the temporomandibular joint and temporal regions. In an alternative embodiment, a device comprises a fabric layer designed to contact the maxillomandibular region, an inflatable layer, a hard plate placed on said fabric layer to resist bulging of said inflatable layer when inflated, and a conductive layer covering said inflatable layer. Methods to use the device to treat the maxillomandibular region and temporomandibular joint of a patient are also disclosed.

Rich, U.S. 2001/0051820 describes a thermal treatment pack and corresponding retainer apparatus that provide not only a desirable thermal source, but also a highly configurable retainer apparatus. According to one embodiment, the thermal source includes frozen peas stored in transparent bags. The transparent bags are selectively attachable through hook-and-loop fasteners. According to the preferred embodiment, the retainer apparatus includes two washable cloth strips. The cloth strips include hook-and-loop fasteners for attaching the transparent bags to the retainer apparatus.

Our prior art search with abstracts described above teaches: an ice bag holder, an adjustable ice bag harness, a therapeutic bandage with removable hot or cold packs, a postoperative cryokinetic therapy apparatus and method, facial ice packs, a device for therapeutic treatment of the temporomandibular and maxillomandibular region and method for using same, a thermal treatment pack and corresponding retainer member and methods of applying thermal treatment, but does not teach a more simple jaw harness with flexible net fabric and adjustable engagement at the back of the head and neck. The present invention fulfills these needs and provides further related advantages as described in the following summary.

### SUMMARY OF THE INVENTION

The present invention teaches certain benefits in construction and use which give rise to the objectives described below.

As can be seen in the references sited above, the prior art teaches the mounting of supporting straps on the head and face in a more or less vertical manner with the straps passing under the jaw and then upwards around the top of the head as seen in Baker, Brennan, Hubbard et al, and Momtaheni, or to a band that passes around the forehead and upper back of the head, as seen in Rich '820, Keen '536, Bagg et al '512 and Lee et al '046. The prior art does not teach a support for hot and cold packs that extends forward to the chin and therefrom across the cheeks to be secured at the back of the head.

However, it has been found, and is fully described herein, that a facial harness for supporting a hot or cold pack can be mounted differently with distinct advantages, i.e., around the chin and to the lower back of head and neck. A facial harness extends in contact with a portion of the human face and neck and especially contacts the cheek area proximal to the teeth and mouth gum surfaces associated therewith. An approximately rectangular fabric extends between an upper strap and a lower strap across the lower facial areas. The fabric provides pockets for holding hot-cold packs. A pair of straps hold the fabric in place on the face and neck. An elastic piece at the front allows for firm contact with the face. Horizontal and vertical oriented pockets are appropriately placed for thermal contact with certain muscles in the face.

A primary objective of the present invention is to provide an apparatus and method of use of such apparatus that yields advantages not taught by the prior art.

Another objective is to provide such an invention capable of supporting an cold or hot pack against the jaw while allowing for full function of the head and mouth.

A further objective is to provide such an invention capable of holding an cold or hot pack of various sizes over a considerable range of sizes.

A still further objective is to provide such an invention capable of low cost manufacture and compact storage.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective view of the invention; and

FIG. 2 is a left side elevational view thereof as mounted on a face and neck, the right side elevational view being a mirror image thereof.

### DETAILED DESCRIPTION OF THE INVENTION

The above described drawing figures illustrate the invention in at least one of its preferred embodiments, which is further defined in detail in the following description. Those having ordinary skill in the art may be able to make alterations and modifications in the present invention without departing from its spirit and scope. Therefore, it must be understood that the illustrated embodiments have been set forth only for the purposes of example and that they should not be taken as limiting the invention as defined in the following.

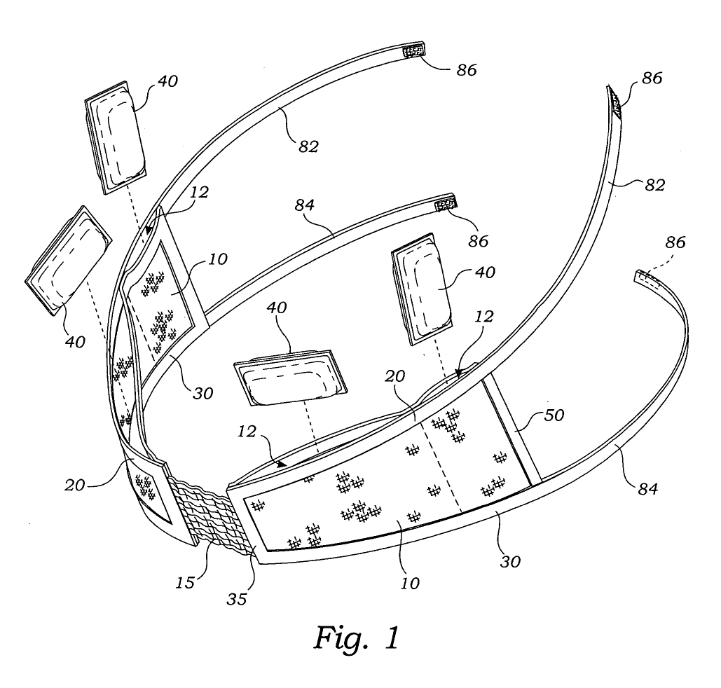

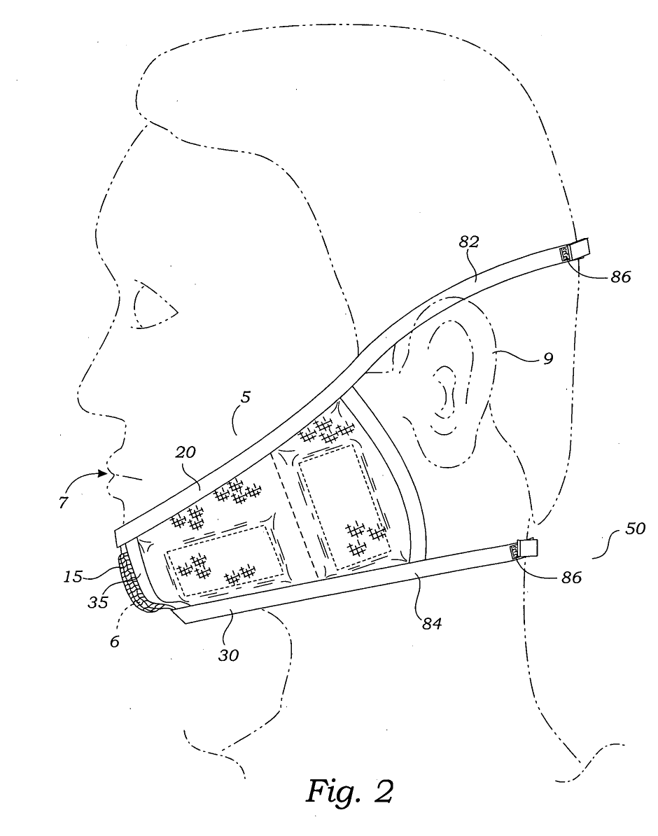

The present invention is a facial harness apparatus as shown in FIG. 1. In use, it is placed on the face of a person as shown in FIG. 2 where it contacts a portion of the human face and neck in a novel way. The apparatus comprises a pair of fabric pocket panels 10 mutually engaged by an elastic strap 15 so that the pocket panels 10 extend in opposing directions. Each of the pocket panes provides an upper edge 20, a lower edge 30 and left 35 and right 50 opposing side edges. Each of the fabric panels 10 is of a size such whereby, the upper edge 20 extends from just below the mouth 7 to a position shy of the ear 9 and the lower edge 30 extends from the chin 6 to about the end of the jaw bone 8 (mandible). Therefore, the pocket panels 10 are wedge shaped. Variation from this shape and size pocket panels 10 is possible while still achieving the objectives of the invention. Finally, the apparatus provides a means for holding 82, 84, 86 the pocket panels 10 on the face, and further for holding the pocket panels 10 in tight contact with the face, as shown in FIG. 2.

Preferably, the holding means is a first 82 and a second 84 holding straps extending rearwardly from each of the pocket panels 10 wherein, preferably, the first one of the holding straps 82 is a continuation of the upper edge 20, and the second one of the holding straps 84 is a continuation of the lower edge 30, as shown in FIG. 2.

Preferably, each of the holding straps 82, 84 provides a fastening means 86 for securing straps 82, 84 behind the head and neck. Alternately, the straps 82, 84 may be of an elastic material to expand so as to fit over the head and neck and thus able to hold the invention in place by elastic tension.

When in place on the face, the pocket panels 10 are joined at the chin by the elastic strap 15 to provide tension for holding the pocket panels 10 securely against the face.

Preferably, the pocket panels 10 are of a size and shape as to receive hot-cold packs, such as blue-ice for applying therapeutic thermal relief for pain or for reducing swelling in the areas covered by the pocket panels 10. Preferably, the pockets 12 are arrange as shown in FIG. 1 so that an ice pack is able to be placed in a forward (closer to the chin) pocket in a horizontal orientation and in a rearward pocket in a vertical orientation. Such placement and orientation of the pockets 12 and thus the hot-cold packs 40, enable thermal therapy to be applied to specific muscles that lay in contact with the pockets 12; specifically the vertically oriented pack 40 in contact with the masseter muscle, and the horizontally oriented pack 40 in contact with the mentalis muscle.

The enablements described in detail above are considered novel over the prior art of record and are considered critical to the operation of the instant invention and to the achievement of the above described objectives. The words used in this specification to describe the invention and its various embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or elements of this described invention and its various embodiments are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments or that a single element may be substituted for two or more elements in a claim.

Changes from the claimed subject matter as viewed by a person with ordinary skill in the art, now known or later devised, are expressly contemplated as being equivalents within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted, and also what essentially incorporates the essential idea of the invention.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. A facial harness apparatus for mounting in contact with a portion of the human race, the face having a chin, a mouth, cheeks, ears and a jaw bone, the apparatus comprising: a pair of fabric pocket panels joined at a front edge of the panels by an elastic strap, the panels extending in opposing directions so that with the elastic strap in contact with the chin an upper edge of each of the panels is directed linearly from below the mouth to immediately above an car, and a lower edge is directed divergently from immediately below the chin to the base of the mandible; the edges further extensive as coextensive straps for fastening the harness around the head as a means for holding the apparatus in place on the face; and a plurality of pockets in each of the panels for engaging hot-cold packs.

2. (canceled)

3. The apparatus of claim 1 wherein the straps provide a strap fastening means for joining the straps for securing the apparatus on the head.

4. (canceled)

5. The apparatus of claim 1 wherein each of the panels issue arranged and sized so that a hot-cold pack is able to be placed in a forward pocket in a horizontal orientation and in a rearward pocket in a vertical orientation.

6. (canceled)

7. (canceled)

8. (canceled)

9. (canceled)

10. (canceled)

11. The apparatus of claim 1 further comprising at least four hot-cold packs, the hot-cold packs engaged within the pockets.

12. The apparatus of claim 11 wherein in each of the panels, one of the hot-cold packs is positioned horizontally in a forward pocket and another of the hot-cold packs is positioned vertically in a rearward pocket.
